(19) United States
(12) Patent Application Publication
(10) Pub. No.: US 2017/0071533 A1
(43) Pub. Date: Mar. 16, 2017

(54) SYSTEMS AND METHODS FOR DETECTING AND DIAGNOSING SLEEP DISORDERED BREATHING

(71) Applicant: HALARE, INC., WARRIORS MARK, PA (US)

(72) Inventors: ANTHONY C. WARREN, WARRIORS MARK, PA (US); MATTHEW MICHAUX, PITTSBURGH, PA (US)

(21) Appl. No.: 15/124,424

(22) PCT Filed: Mar. 13, 2015
(86) PCT No.: PCT/US2015/020388

§ 371 Date: Sep. 8, 2016

Related U.S. Application Data
(60) Provisional application No. 61/952,186, filed on Mar. 13, 2014.
(60) Provisional application No. 61/952,183, filed on Mar. 13, 2014.

(51) Int. Cl. A61B 5/00; A61B 5/145; A61B 5/0205

(52) U.S. Cl. CPC A61B 5/4818; A61B 5/0205; A61B 5/14542; A61B 5/7282; A61B 5/02405

(57) ABSTRACT

Systems and methods for diagnosing sleep disordered breathing in a user are disclosed. The systems include a sensor assembly configured to detect physiological data of a user while the user is asleep. The systems also include an electronic computing device configured to receive the detected data from the sensor assembly, analyze the detected data for at least one sleep disordered event of the user, determine, from the analyzed data and the at least one event, whether the at least one sleep disordered event is indicative of a sleep disordered breathing symptom, and diagnose the user with a sleep disordered breathing condition when the at least one sleep disordered event is determined to be indicative of a sleep disordered breathing symptom. The systems also include an output device configured to communicate the diagnosed sleep disordered breathing condition to the user.

### CROSS-REFERENCE TO RELATED APPLICATIONS

This application is the U.S. national phase application of PCT International Application No. PCT/US2015/020388 filed Mar. 13, 2015, which claims priority to U.S. Provisional Application No. 61/952,186 entitled “Systems and Methods for the Early Detection of Sleep Disordered Breathing Symptoms,” filed on Mar. 13, 2014, and to U.S. Provisional Application No. 61/952,183 entitled “Systems, Methods, and Apparatuses for the Detection of Sleep Disordered Breathing Symptoms,” filed on Mar. 13, 2014, the contents of each are incorporated fully herein by reference.

### FIELD OF THE INVENTION

The present invention relates to detection of sleep disordered breathing symptoms.

### BACKGROUND OF THE INVENTION

A significant portion of sleep disordered breathing is a condition characterized by repeated episodes during sleep resulting in many detrimental and detectable effects on a person. Research has shown that sleep disordered breathing can have major short term and long term deleterious impacts. Therefore, there exists a need for improved and accessible systems and methods for detecting sleep disordered breathing in persons.

### SUMMARY OF THE INVENTION

Aspects of the invention include systems for diagnosing sleep disordered breathing in a user. The systems include a sensor assembly configured to detect physiological data of a user while the user is asleep. The systems also include an electronic computing device configured to receive the detected data from the sensor assembly, analyze the detected data for at least one sleep disordered event of the user, determine, from the analyzed data and the at least one event, whether the at least one sleep disordered event is indicative of a sleep disordered breathing symptom, and diagnose the user with a sleep disordered breathing condition when the at least one sleep disordered event is determined to be indicative of a sleep disordered breathing symptom. The systems also include an output device configured to communicate the diagnosed sleep disordered breathing condition to the user.

Further aspects of the invention are directed to methods for diagnosing a user with a sleep disordered breathing. The method includes detecting, with a sensor assembly, physiological data of a user while the user is asleep, analyzing, with a processing unit, the detected physiological data for at least one sleep disordered event of the user, determine, with the processing unit, whether the at least one sleep disordered event is indicative of a sleep disordered breathing symptom, diagnosing the user with a sleep disordered breathing condition when the at least one sleep disordered even is determined to be indicative of a sleep disordered breathing symptom, and outputting, with an output device, information related to the sleep disordered breathing condition.

### BRIEF DESCRIPTION OF THE DRAWINGS

The invention is best understood from the following detailed description when read in connection with the accompanying drawings, with like elements having the same reference numerals. When a plurality of similar elements is present, a single reference numeral may be assigned to the plurality of similar elements with a small letter designation referring to specific elements. Included in the drawings are the following figures:

FIG. 1 is a schematic diagram of a system for diagnosing sleep disordered breathing according to aspects of the invention;

FIG. 2 is a schematic diagram of a sensor assembly in accordance with aspects of the invention;

FIG. 3 is a chart depicting data indicative of desaturation in accordance with aspects of the invention; and

FIG. 4 is a flowchart of steps in a method of diagnosing sleep disordered breathing according to aspects of the invention.

### DETAILED DESCRIPTION OF THE INVENTION

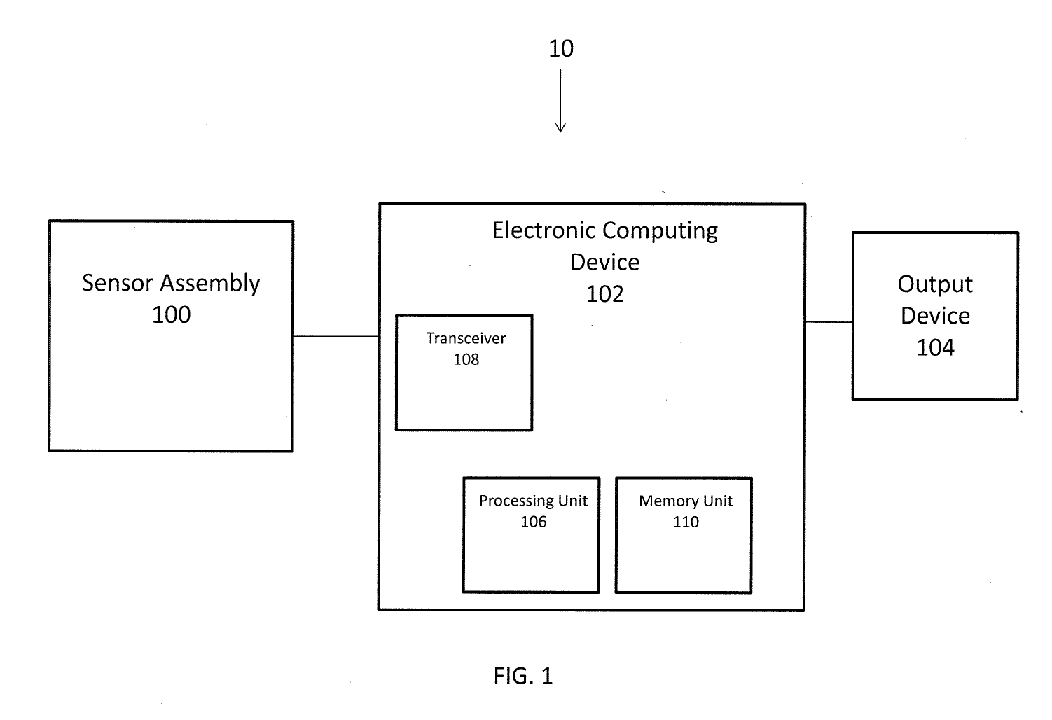

Referring to FIG. 1, a block diagram of a system 10 for diagnosing sleep disordered breathing of a user is shown in accordance with aspects of the invention. The system 10 includes a sensor assembly 100, an electronic computing device 102, and an output device 104. Although the sensor assembly 100, the electronic computing device 102, and output device 104 are depicted as separate components in system 10, it is contemplated that any or all of these components may be integrated together in two or one device. For example, the sensor assembly, the electronic computing device 102, and the output device 104 may be integrated into an apparatus attachable to a user (e.g., a wristband, a neckband, other attachments, etc.), or in a smart device, such as a smart phone, tablet computer, laptop computer, etc.

The sensor assembly 100 includes at least one sensor that is configured to detect physiological data of the user that can be used to detect and diagnose sleep disordered breathing (SDB) of the user. The sensor assembly 100 may include, for example, an accelerometer, a blood oxygen saturation sensor, a motion sensor, an audio sensor, a heart rate sensor, a breath sensor, a position sensor, etc. Other suitable sensors for detecting physiological data of a user will be understood by one of ordinary skill in the art from the description herein.

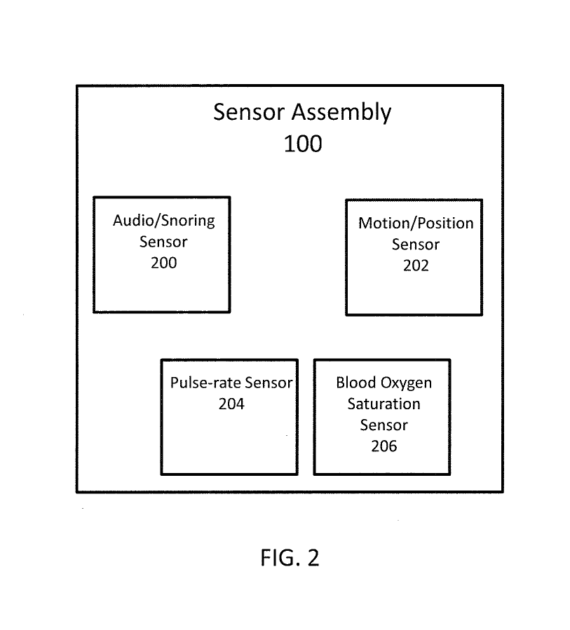

Referring to FIG. 2, a diagram of an example of a sensor assembly 100 is shown. Although the sensor assembly 100 is shown with multiple sensors, the sensor assembly 100 may include only one of the sensors, or any combination of the sensors shown. In one embodiment, the sensor assembly 100 includes an audio/snoring sensor 200 adapted to detect snoring sounds from the user. The snoring sensor 200 may be adapted to detect time intervals between snoring sounds of the user and to detect the intensity of snoring from the user. Analysis of the physiological data in the form of intervals of snoring sounds and intensity of snoring sounds may be used to differentiate between apneic users suffering from Obstructive Sleep Apnea (OSA) and benign persons. Detection of snoring sounds is useful to determine whether a user has a history of snoring, whether snoring is an indication of possible SDB or is generally benign, whether the snoring is an indication of OSA, where in the user airway constrictions are occurring, etc.

Snoring sounds occur when there is an obstruction to the free flow of air through the passages at the back of the mouth and nose. This area is the collapsible part of the airway where the tongue and upper throat meet the soft palate and uvula. Snoring occurs when these structures strike each other and vibrate during breathing. Snoring may be a sign of OSA, a more serious condition. OSA is characterized by multiple episodes of breathing pauses greater than 10 seconds at a time, due to upper airway narrowing or collapse. This results in lower amounts of oxygen in the blood, which causes the heart to work harder. It also causes disruption of the natural sleep cycle, which makes people feel poorly rested despite adequate time in bed. Apnea patients may experience 30 to 300 such events per night.

The sensor assembly 100 may also include a position/movement sensor 202. In one embodiment, the position/movement sensor 202 is adapted to detect restlessness in sleep of the user as an indicator of periods of apnea or hypopnea in a user. In one example, OSA symptoms often exacerbate when the user lies on their back. Periods of excessive movement of the user during sleep may also be detected by the position/movement sensor 202, such that timing and intensity of the restlessness of the user may be analyzed to detect sleep disordered events for further analysis.

The sensor assembly 100 may also include a pulse-rate sensor 204 for detecting the heart rate of the user during sleep. The pulse-rate sensor 204 may be configured to detect periods of elevated pulse rate of the user during sleep, as well as disturbed or erratic pulse rates of the user as sleep disordered events, which may be used for further analysis.

A blood oxygen saturation (O₂Sat) sensor 206 may also be included in the sensor assembly 100. In an embodiment, the O₂Sat sensor 206 is adapted to detect changes in oxygen concentration levels of the user's blood during sleep as potential sleep disordered events.

Referring back to FIG. 1, the electronic computing device 102 includes a processing unit 106, a transceiver 108, and a memory unit 110. The transceiver 108 may be utilized to receive physiological data detected from the sensor assembly 100. In embodiments where the electronic computing device 102 is integrated with the sensor assembly 100, the transceiver 108 may not be a necessary component for the transmission and reception of data to be analyzed by the electronic computing device 102. The memory unit 110 is depicted as integrated into the electronic computing device 102. It is contemplated that additional memory units may be utilized, such as a memory unit integrated into the sensor assembly 100 or a cloud storage device. Such memory units are configured to store detected physiological data and subsequent analyzed data.

The processing unit 106 is adapted to process the data detected by the sensor assembly 100 according to particular algorithms to detect sleep disordered events, determine whether sleep disordered events are indicative of SDB symptoms, and to diagnose the user with a particular sleep disordered breathing disorder when the sleep disordered events are determined to be indicative of SDB symptoms rather than being benign events.

The particular algorithms the processing unit 106 applies to the physiological data detected by the sensor assembly 100 depends upon the type of data detected and the sensors that are used to detect the data. Although the algorithms described herein are related to an individual sensor type, the physiological data analyzed from each type of sensor may be used in conjunction with or in combination with data from other sensors to detect sleep disordered events and SDB symptoms.

Previous systems are unable to reliably diagnose apnea and hypopnea periods and events and other symptoms of SDB without one or both Type I (e.g., an error in detecting an effect that is not present) and Type II errors (e.g., an error failing to detect an effect that is present). For example, the use of O₂Sat may not detect short apnea events as breathing may recommence before the level of blood oxygen has declined sufficiently to be detected. The use of snoring sound analysis may not detect apnea symptoms in patients with a low body mass index or in the case of central nervous apnea/hypopnea. Further the response of physiological parameters may differ over a large range both for a specific individual and between individuals. For this reason, reliance on a single, or a few sensed physiological parameters is insufficient to provide a reliable detection of SDB including apnea and hypopnea periods and events for large and diverse populations.

In examples where the sensor assembly 100 includes an audio/snoring sensor, such as sensor 200, the processing unit 106 may apply algorithms as follows. Baselines may be established for time intervals between snoring of the user and intensity of the snoring for the user. The processing unit 106 receives the snoring physiological data and tracks the snoring of the user. When the snoring sensor 200 records snoring occurrences of the user that occur within a time interval that exceeds the time interval baseline, the processing unit 106 determines that the exceeding of the baseline is a sleep disordered event. Alternatively, the time interval baseline may be established such that the processing unit 106 determines a sleep disordered event if snoring occurrences of the user occur too quickly within one another. Similarly, the audio level of the snoring (e.g., a decibel level) may be indicative of intensity of the snoring of the user. A baseline may be established such that when the audio level of a snoring occurrence of the user exceeds the baseline, the intensity of such snoring occurrence may be determined to be a sleep disordered event by the processing unit 106.

In order to record audible snoring sounds with sufficient fidelity to enable the analysis, the sound sensor 200 may be mounted closer to the face of the user, using, as one example, a microphone near the throat. In this case, the sound sensor 200 may be in a separate sensor module apparatus which communicates either by wire, or preferably wirelessly, to either the wrist mounted module, or directly to an electronic computing apparatus for subsequent analysis as described above. This method increases the fidelity of the audible sound detection by reducing interference say from another snorer nearby, or by a wrist mounted sound detector being physically obscured from the user's head.

In examples where the sensor assembly 100 includes a motion or position sensor 202, baselines may be established against which the physiological data from the motion/position sensor 202 is analyzed with the processing unit 106. Amounts of motion or changes of position of the user during sleep are detected by the motion/position sensor 202. When the amounts of motion or changes of position exceed the established baselines, the processing unit 106 determines that the amount of motion or change of position can be a sleep disorder event. Data from the sensor 202 are analyzed using a rolling average and periods of extensive movement from an established resting baseline and lasting more than 10 seconds indicate periods of restlessness the timing and intensity of which are stored together with the time stamp for subsequent correlation. Similarly, position orientation is also stored with a time stamp for subsequent correlation.

Similarly with a pulse rate sensor 204, the processing unit 106 may be adapted to detect heart rate variability (HRV) in the user during sleep. Analysis techniques, such as converting R-R data sets into frequency domain using Fast Fourier Transform algorithms can derive HRV and the time when the power and frequency spectrum of the HRV change. In addition, longer periods of significantly disturbed or erratic HRV may indicate periods of an intensive series of apnea or hypopnea events. The times, amplitudes, and other associated parameters analyzed from the pulse rate sensor 204 may be used for later correlation analysis.

When an O₂Sat sensor is used, the data detected may be analyzed using a rolling average to seek reductions in the index from a baseline value. Then the processing unit 106 may use a Nervus Algorithm (NA) to extract further detail from the initially determined indications of the occurrence of an apnea or hypopnea event. Typically desaturation is considered to have commenced as soon as the oxygen concentration level falls below the baseline by a specified amount, say by 2% and continues until the signal recovers to a second level which is lower than the baseline by a further 25% of the first determined baseline value. This algorithm defines different levels of drop for desaturation (drop gap) and re-saturation (return gap). This removes any errors that may be associated in assuming the pre- and post-saturation levels are the same. Indeed, often the post-event level lies below the pre-event value.

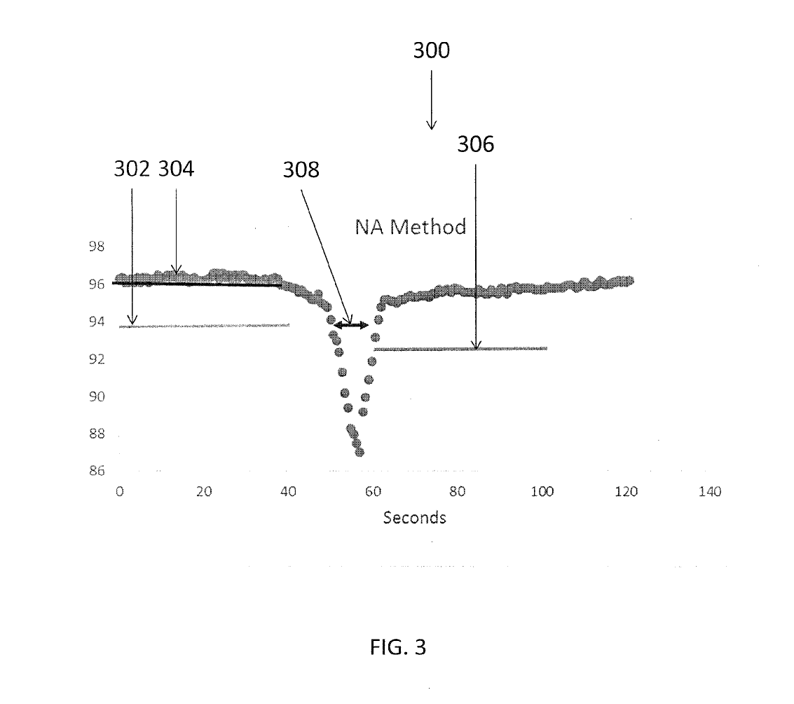

The NA method is shown in the chart 300 of FIG. 3. In this example, the drop gap 302 is arbitrarily taken as 2% below the pre-event baseline 304 and the line 306 is the post-event base-line level which is an additional 0.2% below the pre-event baseline. For this event, the time interval of standard desaturation shown by the double-headed arrow 308, and the lowest value of O₂Sat are recorded. Together they characterize the severity of the apnea event. The desaturation time span and the depth of desaturation for each event are stored together with the time stamp for subsequent correlation.

Thus, the data detected from the sensor assembly 100 may be analyzed by the processing unit 106 in conjunction with each sensor of the sensor assembly 100. Prior to analysis, the data may be pre-processed to remove outliers and long-term trends. Using rolling averaging algorithms on the results of the pre-processed data, periods of brachycardia are determined. The times when such brachycardia events are detected may be used for later correlation analysis.

Further analysis may include, for example, analyzing data from the snoring sensor 200 in conjunction with data from the motion/position sensor 202 to more reliable determine whether a sleep disordered event is caused by a symptom of SDB or is merely benign. More than one indication of a sleep disordered event or symptoms of SDB may be detected in embodiments where multiple or different types of sensors are used in sensor assembly 100. Correlation between more than one indication of a sleep disordered event improves the overall accuracy of the measurements, thereby reducing both Type I and Type II errors.

Along with each data point detected from the sensors of the sensory assembly 100, the system 10 may also include an electronic clock to associate times along with the physiological data detected by the sensor assembly 100. Thus, time signatures can be associated with each data point, which allows for better correlation between data detected from multiple sensors in the sensor assembly 100. In one example, data from an audio sensor (e.g., audio sensor 200) is analyzed using wavelet bi-coherence methods to determine whether periods of snoring are more likely to result from obstructive sleep apnea rather than being benign. The intensity and time span of the snoring events together with their time stamp of occurrence are used for subsequent correlation.

The results of the analysis conducted by the processing unit 106 are subjected to one or more known statistical methods for multivariate data analysis such as so-called principal components, factor, cluster, and discriminant techniques. The correlation of data from multiple independent sensors, each of which has singly demonstrated the ability to diagnose apnea and hypopnea symptoms, significantly reduces errors of both Type I and Type II.

Advantageously, the systems, methods, and apparatus disclosed herein allow for correlation between multiple types of physiological data detected to increase the reliability of the data, reduce the appearance of errors in the data, and more effectively diagnose sleep disordered breathing by determining whether a sleep disordered event is benign or is indicative of a sleep disordered symptom.

In one example, an accelerometer is used to detected restlessness of a user. The restlessness may be detected as compared with a baseline stationary posture ΔR (e.g., non-movement) and may be detected according to an amount of time of restlessness tΔR.

In another example, a blood oxygen saturation sensor is used to detect a period of oxygen desaturation from a pre-determined baseline. A baseline may be, for example, defined as for X seconds below Y % of oxygen saturation. Times of a sleep disordered event from the blood oxygen saturation sensor tSp may be determined from desaturation or oxygen concentration of the blood of the user.

A microphone or audio sensor may also be used to detect snoring sounds. Data detected from a microphone may include amount of snoring sounds, length of snoring events, frequency components, intensity, loudness compared with non-snoring episodes, etc. In one example for loudness compared with non-snoring episodes, three levels of sound (e.g., greater than 30 dB, greater than 40 dB, greater than 50 dB) may be used, and each time the level of sound that occurs from a snoring event exceeds one, or any of the levels of sound tΔSn_{1,2,3} are recorded.

Position of the user may also be detect, where the time the user is laying on his or her back tB is detected and recorded as physiological data for later correlation and diagnosis.

In another example, a pulse-rate sensor is used to detect variation in interbeat intervals. As described above, this physiological data can be used to determine heartrate variability (HRV). The times where the HRV increases from a pre-determined baseline tΔHR are used for correlation and analysis.

The above described physiological data may be detected from the sensor assemblies and then correlated with each other to determine whether a sleep disordered event is indicative of a sleep disordered symptom or is benign. For example, when a snoring event tΔSn is detected that exceeds a baseline level, if it is also detected that the position tB of the user is that of the user laying on his or her back, the detected snoring event is likely benign. Furthermore, if the snoring event is coupled without blood oxygen saturation tSp outside of the baseline and/or without HRV tΔHR outside of the baseline, then the correlation of the data would indicate that the snoring event is benign. The data from the position sensor, the blood oxygen saturation sensor, and the pulse rate sensor may be correlated with the data from the audio sensor to indicate that a snoring event is benign.

Other events outside of snoring may be indicative of sleep disordered breathing symptoms. For example, when the physiological data indicates that tΔHR for HRV exceeds the baseline along with the tSp for blood oxygen levels exceeding the baseline, the system determines that the event detected by tΔHR is that of a sleep disordered breathing event. In another example, when the time of restlessness tΔR exceeds the baseline stationary posture ΔR, but occurs without a registered snoring event tΔSn and/or with the position sensor indicating that the user is not laying on his or her back, the system determines that the event detected by tΔR is indicative of a sleep disordered breathing event.

Sleep-disordered breathing (SDB) often occurs in combination with snoring. In this case they are said to be co-morbid. However SDB may also occur without snoring symptoms being detected. When snoring is associated with the occurrence of sleep-disordered breathing it is no longer necessarily benign. Therefore it is important to determine whether the snoring events are correlated with the occurrence of sleep disordered events such as apnea interruptions. If such correlations are determined the snoring is no longer considered benign.

The systems, methods, and apparatus may be used to detect such co-morbidity of sleep disordered events and snoring events. For example, when a snoring event is detected tΔSn along with a HRV event tΔHR, and/or an oxygen saturation event tSp, and/or a restlessness event tΔR, co-morbidity of snoring and sleep disordered events may exist and should be treated as such. Such co-morbidity is more likely to occur in correlation with apnea events when the apnea is caused by airway restrictions and classed as obstructive sleep apnea (OSA). If the apnea events are a result of disruptive breathing caused by anomalies in the breathing control centers in the central nervous system then it is less likely that any snoring events correlate with the apnea events and they may then be classified as benign.

The system 10 further includes an output device 104. The output device 104 is configured to output sleep disordered events, sleep disordered breathing symptoms, and/or diagnoses to the user or to other relevant personnel (e.g., a doctor, a proctor of an experiment, etc.). The output device 104 may be any device capable of communicating information regarding the analyzed data to a user or other personnel, such as a display of a smart phone, tablet computer, laptop computer, etc. The output device 104 may also be configured to communicate instructions to the user or the personnel for correcting detected sleep disordered breathing symptoms and/or diagnosed sleep disordered breathing. The output device 104 may also be adapted to transmit the information to a doctor, and provide suggested treatments for the user based on the analyzed data.

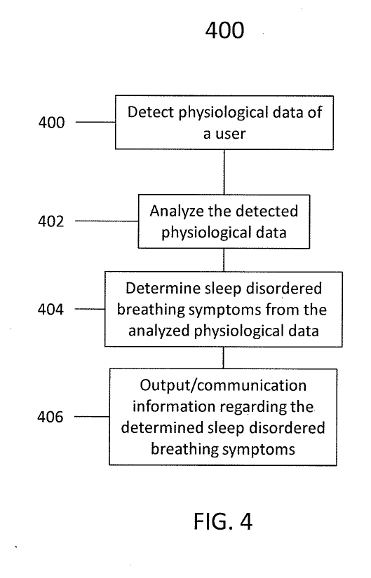

FIG. 4 is a flowchart 40 of steps for detecting sleep disordered breathing according to aspects of the invention. At step 400, physiological data of the user is detected. The data may be detected with a sensor assembly, such as sensor assembly 100. The data may include motion data, position data, audio data, heart rate data, blood oxygen saturation data, etc.

At block 402, the data are analyzed. The data may be analyzed by a processing unit, such as processing unit 106. The data are analyzed to determine whether a sleep disordered event has occurred. Sleep disordered events may occur when the data include data points that exceed or are otherwise outside of a predetermined baseline that is established for the particular type of physiological data detected.

At block 404, sleep disordered breathing symptoms are determined from the analyzed data and the occurred sleep disordered events. Sleep disordered breathing symptoms may be determined by correlating data from multiple sensors. As described above, various sensors and physiological data may be correlated and/or otherwise combined to improve the accuracy of the diagnoses and differentiate between benign sleep disordered events and sleep disordered events that are indicative of sleep disordered breathing symptoms.

At block 406, information regarding the sleep disordered breathing symptoms are outputted/communicated. The sleep disordered breathing symptoms may be communicated via a display on an output device, such as a mobile smart phone, tablet, or computer. The information may be in the form of breathing instructions to the user. In an embodiment, the information is transmitted to a doctor or other personnel in charge of the care of the user, and the information may contain graphical representations of the detected data and indications of the sleep disordered events. Furthermore, the information may also include recommended courses of action to take to improve the sleep of the user or to reduce the number of sleep disordered events that are indicative of sleep disordered breathing symptoms.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A system for diagnosing sleep disordered breathing in a user, comprising:
a sensor assembly configured to detect physiological data of a user while the user is asleep;
an electronic computing device configured to:
receive the detected data from the sensor assembly;
analyze the detected data for at least one sleep disordered event of the user;
determine, from the analyzed data and the at least one event, whether the at least one sleep disordered event is indicative of a sleep disordered breathing symptom; and
diagnose the user with a sleep disordered breathing condition when the at least one sleep disordered event is determined to be indicative of a sleep disordered breathing symptom; and
an output device configured to communicate the diagnosed sleep disordered breathing condition to the user.

2. The system of claim 1, wherein the sensor assembly includes at least one of a blood oxygen saturation sensor, an audio sensor, a pulse rate sensor, a motion sensor, and a position sensor.

3. The system of claim 1, wherein the sleep disordered breathing symptom includes apnea or hypopnea.

4. The system of claim 1, wherein the sensor assembly is configured to be attached to the user.

5. The system of claim 1, wherein the at least one event includes disturbed or erratic heart-rate variability.

6. The system of claim 1, wherein the electronic computing device is further configured to determine whether the at least one sleep disordered event is indicative of a sleep disordered breathing symptom by determining whether the at least one sleep disordered event is benign.

7. The system of claim 1, wherein the at least one event includes a period of restlessness of the user during sleep that exceeds a baseline resting period.

8. The system of claim 1, wherein the at least one event includes a period of snoring of the user during sleep that exceeds a baseline snoring period or an intensity of snoring of the user during sleep that exceeds a baseline snoring intensity.

9. The system of claim 1, wherein the at least one event includes an interval of desaturation, detected by a blood oxygen saturation sensor of the sensor assembly, that exceeds a baseline desaturation time or a baseline desaturation level.

10. A method for diagnosing sleep disordered breathing in a user, comprising the steps of:
detecting, with a sensor assembly, physiological data of a user while the user is asleep;
analyzing, with a processing unit, the detected physiological data for at least one sleep disordered event of the user;
determine, with the processing unit, whether the at least one sleep disordered event is indicative of a sleep disordered breathing symptom;
diagnosing the user with a sleep disordered breathing condition when the at least one sleep disordered even is determined to be indicative of a sleep disordered breathing symptom; and
outputting, with an output device, information related to the sleep disordered breathing condition.

11. The method of claim 10, wherein the detecting step further comprises detecting blood oxygen saturation levels with a blood oxygen saturation sensor.

12. The method of claim 11, wherein the analyzing step comprises analyzing the detected data for desaturation as the at least one sleep disordered event.

13. The method of claim 10, wherein the analyzing step comprises analyzing the detected data for snoring of a user that exceeds a snoring level baseline as the at least tone sleep disordered event.

14. The method of claim 10, wherein the analyzing step comprises analyzing the detected data for periods of restlessness of the user that exceeds a motion or position baseline as the at least one sleep disordered event.

15. The method of claim 10, wherein the determining step comprises correlating data from multiple sensors of the sensor assembly for determining whether the at least one sleep disordered event is indicative of the sleep disordered breathing symptom.
